(19) United States
(12) Patent Application Publication
(10) Pub. No.: US 2015/0328052 A1
(43) Pub. Date: Nov. 19, 2015

(54) Ear Protection Device and Assembly Method

(71) Applicant: A.J. Kalden Beheer en Management B.V., Huizen (NL)

(72) Inventor: Arnoud Jan Kalden, Huizen (NL)

(21) Appl. No.: 14/404,752

(22) PCT Filed: May 31, 2013
(86) PCT No.: PCT/NL2013/050393

§ 371 Date: Dec. 1, 2014

(30) Foreign Application Priority Data
May 31, 2012 (NL) 1039637

(51) Int. Cl. A61F 11/08

(52) U.S. Cl. CPC A61F 11/08; A61F 2011/085; A61F 2220/0033; A61F 2250/006; A61F 2250/0039; A61F 2250/0075

(57) ABSTRACT

A hearing protective device placeable in an auditory canal of an ear, includes: a first outer end extending during use into the auditory canal, a second outer end remote from the first outer end and configured for gripping during arranging and/or removing of the hearing protective device respectively into and/or out of the auditory canal, a body extending between the first and the second outer end and provided with a channel for the passage of sound, a filter space arranged in the body for placing of a sound damping filter, and at least one sealing member which is connected by means of a base to the body for the purpose of blocking sound in the auditory canal during use. The filter space is arranged in the body such that, as seen in side view in the longitudinal direction of the central axis of the channel, it at least overlaps at least a part of the sealing member.

A first aspect of the invention relates to a hearing protective device placeable in an auditory canal of an ear, the device being provided with:
a first outer end extending during use into the auditory canal,
a second outer end remote from the first outer end and configured for gripping during arranging and/or removing of the hearing protective device respectively into and/or out of the auditory canal,
a body extending between the first and the second outer end and provided with a channel for the passage of sound.

A second aspect of the invention relates to a method for assembling a hearing protective device.

Said hearing protective device is generally known in the field and is described particularly in NL9401212. The described hearing protective device has a sealing member in the form of a substantially spherical flexible lamella which, when it is placed in the auditory canal, is intended to fit against a peripheral wall of the auditory canal in order to block entry of ambient sound to parts located further into the ear, such as the auditory nerves in the inner ear. The described hearing protective device can be equipped with a sound damping filter in a grip arrangeable relative to the body and configured for gripping by a user in order to be arranged in and/or removed from the auditory canal.

A drawback of such devices is that they provide insufficient protection for the parts located further into the ear relative to the auditory canal, such as auditory nerves in an inner ear.

It is an object of the invention to provide a hearing protective device which during use provides an improved protection of the parts located in the ear from ambient sound harmful to the parts located in the ear.

The device according to the invention is a hearing protective device according to the preamble which is provided for this purpose with:
a filter space arranged in the body for placing of a sound damping filter, and
at least one sealing member which is connected by means of a base to the body for the purpose of blocking sound in the auditory canal during use, wherein the filter space is arranged in the body such that, as seen in side view in the longitudinal direction of the central axis of the channel, it at least overlaps at least a part of the sealing member. Protection of the parts located further into the ear, such as the auditory nerves in the inner ear, hereby becomes possible during use, this protection being aimed at preventing exposure of the parts located further into the ear to ambient sound harmful to these parts, wherein the sealing member is connected to the body such that the ambient sound can be guided into the auditory canal at least partially via a filter placed in the filter space, and wherein the ambient sound which has access along an outer side of the body to the parts located further into the ear can be reduced.

In an embodiment the filter space is arranged in the body such that, as seen in side view in the longitudinal direction of the central axis of the channel, it at least partially overlaps the base. It hereby becomes possible for the filter placeable in the filter space to extend deep into the auditory canal during use, wherein dimensions of the filter are so large compared to dimensions of the auditory canal that the filter forms a considerable obstacle to passage of ambient sound.

In an embodiment the base is situated between a first filter space end facing toward the first outer end and a second filter space end facing toward the second outer end. It hereby becomes possible for the filter placeable in the filter space to extend deep into the auditory canal during use, wherein dimensions of the filter are so large compared to dimensions of the auditory canal that the filter forms a considerable obstacle to passage of ambient sound.

In an embodiment the sealing member comprises a first lamella, wherein the filter space is arranged in the body such that, as seen in side view in the longitudinal direction of the central axis of the channel, it at least overlaps at least a part of the first lamella. It hereby becomes possible to absorb the ambient sound by means of the first lamella and substantially expose the filter placeable in the filter space thereto for the purpose of achieving an improved damping.

In an embodiment the first lamella extends in the direction toward the second outer end to a position beyond the second filter space end. It hereby becomes possible to absorb a part of the ambient sound to which the ear is exposed and to concentrate it toward the filter placeable in the filter space for the purpose of achieving an improved damping.

In an embodiment the sealing member comprises at least a second lamella, wherein the second lamella extending between the first lamella and the first outer end is connected to the body. It hereby becomes possible to absorb by means of the second lamella a part of the ambient sound passing beyond the first lamella for the purpose of damping.

In an embodiment the filter space is arranged in the body such that, as seen in side view in the longitudinal direction of the central axis of the channel, it at least overlaps at least a part of the second lamella. It hereby becomes possible to absorb by means of the second lamella a part of the ambient sound passing beyond the first lamella and to substantially still expose the filter placeable in the filter space thereto for the purpose of achieving a further improved damping.

In an embodiment the sealing member comprises a further lamella, wherein the further lamella extending between the second lamella and the first outer end is connected to the body. It hereby becomes possible during use with the further lamella to provide a part of the hearing protective device extending deepest into the ear with a sealing means for blocking sound in the auditory canal.

In an embodiment the sealing member comprises a third lamella, wherein the third lamella extending between the second lamella and the first outer end is connected to the body. It hereby becomes possible during use with the further lamella to provide a part of the hearing protective device extending deepest into the ear with a sealing means for blocking sound in the auditory canal.

In an embodiment the sealing member comprises a further lamella, wherein the further lamella extending between the first lamella and the second outer end is connected to the body. It hereby becomes possible during use with the further lamella to provide a part of the hearing protective device extending least deeply into the ear with a sealing means for blocking sound in the auditory canal.

In an embodiment the sealing member has substantially a flange shape. A sealing member is hereby obtained which during use in an auditory canal fits snugly against a peripheral wall of the auditory canal.

In an embodiment the sealing member extends outwardly out of the body from the base, preferably in a direction from the first outer end, more preferably with a curvature substantially similar to a partial spherical shape. It hereby becomes possible for the ambient sound to be at least partially absorbed during use and directed and/or guided toward the filter placed in the filter space for the purpose of damping as much ambient sound as possible to which the ear is exposed.

In an embodiment the channel extends through the filter space. It hereby becomes possible that the ambient sound to which the ear is exposed and which has access by means of the channel to parts located further into the ear, such as the auditory nerves in the inner ear, is forced through the filter placed in the filter space for the purpose of damping the sound.

In an embodiment a sound damping filter is placed in the filter space. It hereby becomes possible that the ambient sound to which the ear is exposed and which has access by means of the channel to parts located further into the ear, such as the auditory nerves in the inner ear, can be filtered by the filter placed in the filter space for the purpose of damping the sound.

In an embodiment the filter space is situated at a second distance from the second outer end. It hereby becomes possible for the filter placeable in the filter space to extend during use into the auditory canal.

In an embodiment the second distance is substantially equal to, or the second distance is at least greater than, a distance between the second outer end and a part of the sealing member facing toward the second outer end. It hereby becomes possible for the filter placeable in the filter space to extend deep into the auditory canal during use, wherein dimensions of the filter are so large compared to dimensions of the auditory canal that the filter forms a considerable obstacle to the passage of ambient sound.

In an embodiment the filter space has a first diameter which is preferably substantially constant as seen in the longitudinal direction. It hereby becomes possible during use to position a filter with a constant outer diameter in an auditory canal, which filter becomes relatively larger compared to a diameter of the auditory canal as an auditory canal becomes narrower in the direction toward the parts located further into the ear.

In an embodiment the body comprises a part facing toward the second outer end, wherein the channel has close to the filter space a second diameter which is smaller than the first diameter of the filter volume. It is hereby possible, after placing of the filter in the filter space, to hold the filter properly at this position during use.

In an embodiment the part of the channel facing toward the second outer end has close to the second outer end a third diameter which is greater than the second diameter for the purpose of guiding a filter during displacement from the second outer end to the filter space. It hereby becomes possible to displace the filter easily through the channel to the filter space.

In an embodiment a first filter space end facing toward the first outer end has a stop for receiving and/or holding the filter in a direction and/or position directed furthest toward the first outer end relative to the body. It hereby becomes possible to position the filter during placing in the filter space.

In an embodiment the stop comprises a surface which extends substantially transversely of the central axis of the channel. It hereby becomes possible during removal of the hearing protective device from an auditory canal for a pulling force on the second outer end to be converted to a pressing force on the filter for the purpose of co-displacing the filter, wherein the pressing force is distributed over the surface extending substantially transversely of the central axis of the channel.

In an embodiment the body and the sealing member are manufactured from a material in a single piece, and/or wherein the material comprises a thermoplastic elastomer. This prevents the presence of a weakened connection between the body and the sealing member, wherein the number of separate components of the hearing protective device is considerably limited.

A second aspect according to the invention relates to a method for placing a sound damping filter in a filter space of a hearing protective device, comprising the steps of:
inserting the sound damping filter into a channel via the second outer end of the hearing protective device, and
displacing the sound damping filter through the channel toward the filter space until the sound damping filter is located in the filter space. It hereby becomes possible to assemble a hearing protective device, wherein the filter is displaced through the channel over at least a second distance extending between the second outer end and the second filter space end for the purpose of placing the filter in the filter space, wherein the filter is enclosed by the sealing member, and wherein the filter extends during use deep into the auditory canal and wherein the filter is confined in a secure manner in the body of the hearing protective device. Despite the fact that the measure wherein the filter is displaced over the second distance through the channel is not obvious for labour-intensive reasons, this method provides the possibility of providing a hearing protective device according to the invention.

The invention will now be described in more detail on the basis of an embodiment and with reference to the figures, in which:

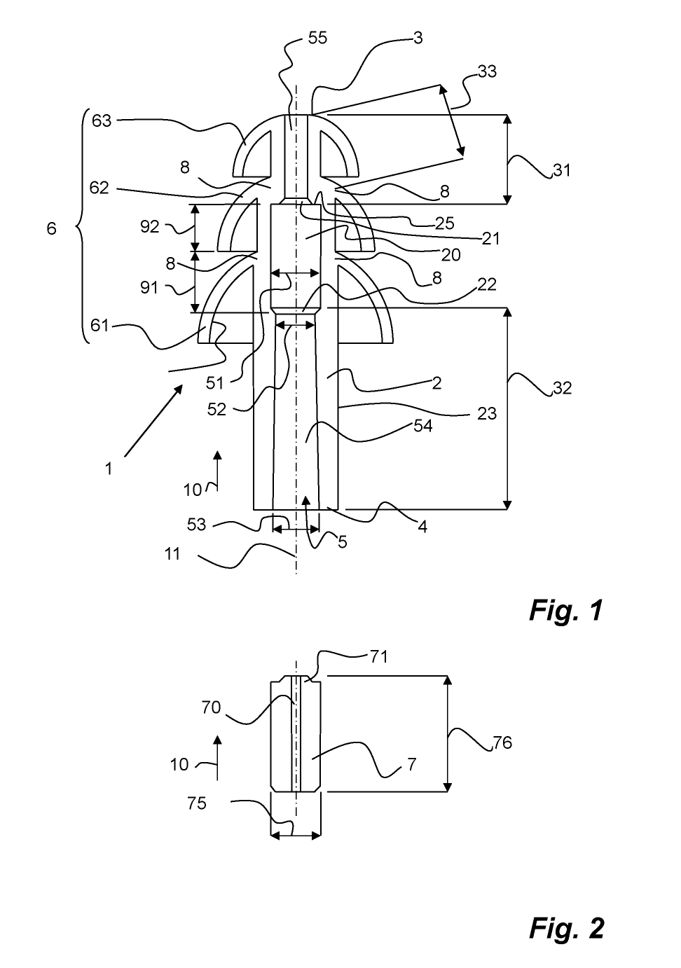

FIG. 1 shows a cross-section along the longitudinal direction of a hearing protective device according to an embodiment of the invention,

FIG. 2 shows a cross-section of a filter placeable in a filter space formed by the widening as according to FIG. 1.

The same reference numerals designate the same components in all figures. FIG. 1 shows an embodiment of a hearing protective device 1 according to the invention, this hearing protective device 1 being substantially universally applicable, i.e. the hearing protective device 1 is suitable for use by a large group of people, wherein the hearing protective device 1 is suitable for arrangement in practically any ear. An advantage of such a hearing protective device 1 is that dimensions of the external parts, such as sealing member 6, need not be modified to a shape and dimensions of an auditory canal of a user.

The hearing protective device 1 is of substantially elongate form, extends in a longitudinal direction 10 along a central axis 11, wherein the shown cross-section is arranged about the central axis 11. The hearing protective device 1 has a first outer end 3 which extends during use into the auditory canal, and a second outer end 4 remote from the first outer end 3 and configured for gripping during arranging and removal of the hearing protective device 1 respectively into and/or out of the auditory canal. In this embodiment second outer end 4 is grip-like and forms part of a body 2 which extends between the first and the second outer ends 3, 4 and which is provided with a channel 5 for passage of sound and/or sound waves.

Body 2 is substantially elongate, wherein an outer peripheral wall 23 of body 2 takes a substantially cylindrical form in this embodiment. Body 2 can have any desired form instead of the cylindrical form, and for instance be angular and/or polygonal, such as triangular, square and so on, wherein the polygonal body provides an improved gripping.

Channel 5 is a continuous opening extending between the first and second outer end 3, 4 of the hearing protective device 1. FIG. 1 shows a filter space 20 arranged in channel 5 of body 20 for the purpose of placing in channel 5, or at least in a part 20 of channel 5, a filter 7 as shown in FIG. 2 and intended for damping sound, such as sound which is harmful to parts located further into the ear, such as the auditory nerves in the inner ear. Channel 5 with the filter 7 arranged therein is configured to damp sound, preferably in a differentiated manner wherein ambient sound is damped more than specific frequency parts of voice sound.

The filter 7 shown in FIG. 2 is in this embodiment a per se known filter 7 which has an outer diameter 75 in a range of 1 to 10 mm, preferably 3 to 4 mm, and particularly equal to 3.1 mm. A passage 70 extends through filter 7 in the longitudinal direction 10 of filter 7 wherein, in order to allow damped passage of harmful sound and to allow sufficient passage of voice sound, the passage 70 has a very small cross-sectional diameter in a range of 0.1 to 0.4 mm, and wherein filter 7 has a length dimension 76 lying in the range of 2 to 10 mm, preferably 4 to 8 mm, and more preferably 6 to 7 mm, and in particular equalling 6.5 mm. Filter space 20 is adapted to the dimensions of filter 7, wherein filter space 20 has a first diameter 51 which, as seen in longitudinal direction 10 is preferably substantially constant and substantially equal to the outer diameter 75 of filter 7, and wherein the filter space 20 has a length dimension substantially equal to the length dimension 76 of filter 7.

Channel 5 comprises a part 55 of substantially straight cylindrical form and having a substantially constant inner diameter, which part 55 extends over a first distance 31 between the first outer end 3 and a first filter space end 21 of filter space 20 facing toward the first outer end 3. Channel 5 comprises a conical part 54 extending over a second distance 32 between the second outer end 4 and a second filter space end 22 of filter space 20 facing toward the second outer end 4 such that a section close to second outer end 4 is greater than a further section close to the second filter space end 22. Channel 5 has a second diameter 52 close to filter space 20 which is smaller than the first diameter 51 of filter volume 20.

The part 54 of channel 5 facing toward the second outer end 4 has a third diameter 53 close to second outer end 4 which is greater than the second diameter 52 for the purpose of guiding a filter 7 during displacement from the second outer end 4 to filter space 20.

The third diameter 53 is smaller than the outer diameter 75 of filter 7. During placing of the filter 7 placeable in filter space 20 the filter 7 is inserted via the second outer end 4 into channel 5 of the hearing protective device 1. The body 2, which is manufactured from a flexible, pliable material such as a thermoplastic elastomer, stretches locally under the influence of the insertion of filter 7, which is manufactured from a harder ceramic material, into the conical part 54, wherein both an inner diameter and an outer diameter of body 2 are increased, at least where filter 7 is located. As filter 7 is displaced through the conical shaft of conical part 54 to filter space 20, channel 5 yields wherein the further section close to the second filter space end 22 is eventually stretched, wherein the second diameter 52 is increased. As soon as filter 7 has been pressed in its entirety through conical part 54, conical part 54 of channel 5 substantially takes on its initial shape and cross-sectional dimension, and filter 7 is located in a confined state in filter space 20.

Relative to a diameter of the straight cylindrical part 55 and conical part 54 of channel 5, the filter space 20 is a widened portion which extends in longitudinal direction 10 with a substantially constant further diameter substantially equal to the outer diameter 75 of filter 7, this outer diameter 75 being larger than the diameter of the straight cylindrical part 55 and conical part 54 of channel 5.

Sealing member 6 is configured to lie during use against a peripheral wall of the auditory canal, wherein under the influence of a clamping action a shape of sealing member 6 is adapted to an extent to a shape of the auditory canal, wherein sealing member 6 is manufactured from a material which is elastic, pliable, flexible and/or compressible, such as a thermoplastic elastomer. During use the hearing protective device 1 hereby remains clamped in the auditory canal to an extent which allows the hearing protective device 1 to be removable from the auditory canal with a normal pulling force on the grip-like second outer end 4 of the hearing protective device 1.

According to the invention the sealing member 6 is connected by means of base 8 to body 2 for the purpose of blocking sound in the auditory canal during use, wherein filter space 20 is arranged in body 2 such that, as seen in side view in the longitudinal direction 10 of central axis 11 of channel 5 as according to FIG. 1, it at least overlaps at least a part 91, 92 of sealing member 6, such as the base 8 of sealing member 6.

In this embodiment the sealing member 6 of the hearing protective device 1 is provided with a first lamella 61, a second lamella 62 and a third lamella 63, wherein first lamella 61 is arranged further from first outer end 3 relative to second lamella 62 and wherein second lamella 62 is arranged further from first outer end 3 relative to third lamella 63. The first, second and third lamellas 61, 62, 63 extend outwardly out of body 2 from base 8, preferably in a direction away from the first outer end 3, more preferably with a curvature substantially similar to a partial spherical shape, wherein lamellas 61, 62, 63 have a flange form or a form similar to an apron.

The third lamella 63 is the part of the hearing protective device 1 which protrudes deepest into the auditory canal during use. Because the auditory canal generally has a narrower passage in the direction of parts located further into the ear, such as auditory nerves in an inner ear, third lamella 63 has a greater curvature than second lamella 62, and second lamella 62 has a greater curvature than first lamella 61.

Base 8 of first lamella 61 is situated between the first filter space end 21 and the second filter space end 22, wherein filter space 20 protrudes through base 8 of first lamella 61. During use ambient sound to which a concave shaped part 60 of first lamella 61 is exposed can be guided to at least a part of the filter 7 placed in filter space 20 for the purpose of damping the ambient sound. Filter 7 placed in filter space 20 allows passage of only damped ambient sound to the parts located further into the ear.

The first filter space end 21 is provided with a stop 25 for the purpose of receiving and/or holding the filter 7 placeable in filter space 20 in a direction and/or position facing furthest toward the first outer end 3 relative to body 2. During removal from the auditory canal of a hearing protective device 1 arranged in the auditory canal a pulling force is exerted on body 2 and is converted by stop 25 to a pressing force on the filter 7 placed in filter space 20. The stop 25 is situated according to the invention close to the base 8 which, during removal of the hearing protective device 1, acts as a reinforcement for the co-displacement of filter 7.

In this embodiment the stop 25 is provided with a surface 25 extending substantially transversely of the central axis 11 of channel 5, wherein an engaging surface 25 is formed against which a filter end 71 of the placeable filter 7 facing toward the first outer end 3 can be pressed.

Body 2 and sealing member 6, such as the first, the second and the third lamellas 61, 62, 63 can be manufactured, for instance by means of injection moulding, from a material in a single piece, wherein the hearing protective device 1 is free of connecting parts, whereby a hearing protective device 1 can be obtained which is safe during use. Assembly is moreover limited to placing of filter 7 in filter space 20.

The hearing protective device 1 according to the invention is manufactured from material comprising a thermoplastic elastomer, wherein a hearing protective device can be obtained which is light, pliable, flexible and strong during use, this material having anti-allergenic properties and being preferred to manufacture from for instance silicone. The sound damping filter 7 can be manufactured from a ceramic material.

In other words, the invention relates to a hearing protective device 1 provided with:
a first outer end 3 which extends during use into the auditory canal,
a second outer end 4 remote from the first outer end 3 and configured for gripping during arranging and/or removal of the hearing protective device 1 respectively into and/or out of the auditory canal,
a body 2 extending between the first and the second outer ends 3, 4 and provided with a channel 5 for transport of sound,
a filter space 20 present in body 2 for the purpose of placing in channel 5 a filter for damping the sound,
a sealing member 6 which is connected by means of a base 8 to body 2 for blocking sound in the auditory canal during use, wherein a first distance 31 extends from first outer end 3 to filter space 20 and wherein a third distance 33 extends from first outer end 3 to base 8, wherein the third distance 33 is substantially equal to the first distance 31, and/or wherein the third distance 33 is at least as great as the first distance 31. Filter space 20 is arranged in body 2 such that it has at least an overlap with a projection of sealing member 6 directed onto the central axis 11 of channel 5.

The invention is of course not limited to the described and shown preferred embodiment but extends to any embodiment falling within the scope of protection as defined in the claims and as seen in the light of the foregoing description and accompanying drawings. The favourable effect of the invention can also be achieved with a single first lamella 61 which has a base 8 through which the filter space 20 protrudes, wherein no further lamellas need be present. The favourable effect of the invention can also be achieved with a plurality of lamellas. Although the shown lamellas 61, 62, 63 take the form of a toadstool or a dish, lamellas 61, 62, 63 can take any form wherein sound is absorbed in an element with a surface of concave form and directed toward the filter 7 placeable in filter space 20.

1-23. (canceled)

24. A hearing protective device placeable in an auditory canal of an ear, the device comprising:
a first outer end extending during use into the auditory canal,
a second outer end remote from the first outer end and configured for gripping during arranging and/or removing of the hearing protective device respectively into and/or out of the auditory canal,
a body extending between the first and the second outer end and provided with a channel for the passage of sound,
a filter space arranged in the body for placing of a sound damping filter, and
at least one sealing member which is connected by means of a base to the body for the purpose of blocking sound in the auditory canal during use, wherein the filter space is arranged in the body such that, as seen in side view in the longitudinal direction of the central axis of the channel, it at least overlaps at least a part of the sealing member.

25. The hearing protective device as claimed in claim 24, wherein the filter space is arranged in the body such that, as seen in side view in the longitudinal direction of the central axis of the channel, it at least overlaps the base.

26. The hearing protective device as claimed in claim 24, wherein the base is situated between a first filter space end facing toward the first outer end and a second filter space end facing toward the second outer end.

27. The hearing protective device as claimed in claim 25, wherein the sealing member comprises a first lamella, and wherein the filter space is arranged in the body such that, as seen in side view in the longitudinal direction of the central axis of the channel, it at least overlaps at least a part of the first lamella.

28. The hearing protective device as claimed in claim 27, wherein the first lamella extends in the direction toward the second outer end to a position beyond the second filter space end.

29. The hearing protective device as claimed in claim 27, wherein the sealing member comprises at least a second lamella, and wherein the second lamella extending between the first lamella and the first outer end is connected to the body.

30. The hearing protective device as claimed in claim 29, wherein the filter space is arranged in the body such that, as seen in side view in the longitudinal direction of the central axis of the channel, it at least overlaps at least a part of the second lamella.

31. The hearing protective device as claimed in claim 29, wherein the sealing member comprises a further lamella, and wherein the further lamella extending between the second lamella and the first outer end is connected to the body.

32. The hearing protective device as claimed in claim 29, wherein the sealing member comprises a third lamella, and wherein the third lamella extending between the second lamella and the first outer end is connected to the body.

33. The hearing protective device as claimed in claim 32, wherein the sealing member comprises a further lamella, and wherein the further lamella extending between the first lamella and the second outer end is connected to the body.

34. The hearing protective device as claimed in claim 24, wherein the sealing member has substantially a flange shape.

35. The hearing protective device as claimed in claim 24, wherein the sealing member extends outwardly out of the body from the base.

36. The hearing protective device as claimed in claim 24, wherein the channel extends through the filter space.

37. The hearing protective device as claimed in claim 24, wherein a sound damping filter is placed in the filter space.

38. The hearing protective device as claimed in claim 24, wherein the filter space is situated at a second distance from the second outer end.

39. The hearing protective device as claimed in claim 38, wherein the second distance is substantially equal to, or wherein the second distance is at least greater than, a distance between the second outer end and a part of the sealing member facing toward the second outer end.

40. The hearing protective device as claimed in claim 24, wherein the filter space has a first diameter which is preferably substantially constant as seen in the longitudinal direction.

41. The hearing protective device as claimed in claim 40, wherein the body comprises a part facing toward the second outer end, and wherein the channel has close to the filter space a second diameter which is smaller than the first diameter of the filter volume.

42. The hearing protective device as claimed in claim 41, wherein the part of the channel facing toward the second outer end has close to the second outer end a third diameter which is greater than the second diameter for the purpose of guiding a filter during displacement from the second outer end to the filter space.

43. The hearing protective device as claimed in claim 24, wherein a first filter space end facing toward the first outer end has a stop for receiving and/or holding the filter in a direction and/or position directed furthest toward the first outer end relative to the body.

44. The hearing protective device as claimed in claim 43, wherein the stop comprises a surface which extends substantially transversely of the central axis of the channel.

45. The hearing protective device as claimed in claim 24, wherein the body and the sealing member are manufactured from a material in a single piece, and/or wherein the material comprises a thermoplastic elastomer.

46. A method for placing a sound damping filter in a filter space of a hearing protective device, comprising the steps of:
inserting the sound damping filter into a channel via a second outer end of the hearing protective device, and
displacing the sound damping filter through the channel toward the filter space until the sound damping filter is located in the filter space.
